(19) United States
(12) Patent Application Publication
(10) Pub. No.: US 2009/0005710 A1
(43) Pub. Date: Jan. 1, 2009

(54) Ultrasonic Equipment for Treatment of Osteoarthritis

(75) Inventors: Byoung-Hyun Min, Gyeonggi-do (KR); Sc-Ra Park, Incheon (KR)

Correspondence Address: INTELLECTUAL PROPERTY / TECHNOLOGY LAW, PO BOX 14329, RESEARCH TRIANGLE PARK, NC, 27709, US

(73) Assignee: DUPLOGEN, INC., Suwon-si (KR)

(21) Appl. No.: 11/576,464

(22) PCT Filed: Sep. 30, 2005
(86) PCT No.: PCT/KR2005/003254

§ 371 Date: Sep. 3, 2008

(30) Foreign Application Priority Data
Oct. 1, 2004 (KR) 10-2004-0078439

(51) Int. Cl. A61N 7/00

(52) U.S. Cl. 601/2

(57) ABSTRACT

The present invention relates to an ultrasonic device for the treatment of arthritis, and more particularly, to an ultrasonic device for the treatment or prevention of degenerative arthritis, which is so structured that it generates ultrasonic waves with an intensity of 200-800 mW/cm2 and applies low-intensity ultrasonic energy of 100-400 mW/cm2 to cartilage cells deep in the skin's dermis. The inventive ultrasonic device comprises: a main control unit including an ultrasonic oscillation control unit allowing the generation of ultrasonic waves with a frequency of 0.1-5 MHz and low intensity of 200-800 mW/cm2; an ultrasonic generator for generating ultrasonic waves according to the control state of the main control unit; an ultrasonic vibrator that vibrates according to ultrasonic waves inputted from the ultrasonic generator; and a probe for transferring ultrasonic vibration to a joint to be treated. The ultrasonic device can induce the regeneration of cartilage tissue by the activation of cartilage tissue, and thus, can fundamentally treat degenerative arthritis.

### TECHNICAL FIELD

The present invention relates to an ultrasonic device for the treatment of arthritis, and more particularly, to an ultrasonic device for the treatment or prevention of degenerative arthritis, which is so constructed that it generates ultrasonic waves with an intensity of 200-800 mW/cm² and applies low-intensity ultrasonic energy of 100-400 mW/cm² to cartilage cells deep in the skin's dermis.

### BACKGROUND ART

Degenerative arthritis is called by various names, including osteoarthritis and senile arthritis, and refers to a disease where the smooth surface of the joint cartilage, which is a bluish-white glittering smooth substance connected to the end of a bone is broken due to the change of the joint cartilage to make an uneven surface, or cause cartilage breakdown in weight-bearing joints over time.

A structure where two or more bones are movably connected to each other is generally called a “joint”, and regions having joints include the knee, the jaw, the shoulder, the hips, a finger, a toe, an elbow, a neck and the vertebra. The bone end of joints is covered with the joint cartilage, and the joint cartilage, which is a tissue comprising cartilage cells and a large amount of matrices surrounding them, serves to prevent the friction of the bone end.

When the joint cartilage is damaged so that cartilage wears out, the bone below the cartilage is abnormally regenerated to cause pain and the abnormality of joints thus, developing degenerative arthritis (osteoarthritis). Namely, the joint cartilage repeatedly undergoes regeneration procedures after being worn out. If factors, such as aging, obesity, articular dysplasia, trauma, some special works, a past history of arthritis, and genetic factors, make the damage of the joint cartilage severe and make its regeneration slow, the bone ends of a joint come in direct contact to cause damage to the bone end, and a bone will be abnormally regenerated, thus causing pain and abnormality.

Therapies which are mainly performed to treat said degenerative arthritis include suitable rest and exercise, the administration of cartilage regenerating agents and analgesics, the injection of joint injection solution, the use of anti-inflammatory agents, debridement, osteotomy, a surgical operation using an arthroscope, and total knee arthroplasty.

However, it was recently known that, when ultrasonic waves are applied to joint regions, ultrasonic vibration would be transferred to cartilage cells within the cartilage tissue to directly influence the activation of the cartilage cells so that degenerative arthritis caused by a reduction in the activity of the cartilage cells could be treated. Due to this fact, ultrasonic devices for the treatment of arthritis are being developed.

In the prior art relating to such ultrasonic devices for treating arthritis using ultrasonic waves, Korean Utility Model Registration No. 20-0296735 discloses an ultrasonic device for the treatment of arthritis, which is so designed that pluralities of ultrasonic elements can adhere closely to severely curved regions, such as the knee, so that ultrasonic waves can sterically act on a knee to increase a therapeutic effect. As shown in FIG. 1, the ultrasonic device disclosed in Korean Utility Model Registration No. 20-0296735 comprises: a plurality of ultrasonic elements that ultrasonically vibrate upon the application of electric power; a support band in which the ultrasonic elements are arranged in a given pattern and detachably assembled on the inner side and which is of such a width that it covers around an arm or a leg; a pair of members for fixing to an arm and hand, which are placed at both ends of the support band, respectively, and detachably fixed to each other; and a female connector which is placed on the surface of the support band and to which an electric wire for applying electric power to the ultrasonic elements is connected and a male connector to be connected to the external power source is coupled. Also, Korean patent No. 10-0461049 discloses an exclusive gel for the ultrasonic treatment of arthritis, which can be used with a device for treating arthritis using ultrasonic waves and is prepared by adding a natural extract with a pain inhibitory or anti-inflammatory effect to a gel for ultrasonic treatment, which is used as a lubricant upon the use of the ultrasonic device, as well as a preparation method thereof.

However, the above-described arthritis treatment devices aim to allow close adherence to severely curved joint regions or to obtain accompanying effects by using separate members capable of increasing the effect of ultrasonic waves, and they have a disadvantage in that they are insufficient as devices for more fundamentally treating arthritis.

### DISCLOSURE OF THE INVENTION

The present inventors have made extensive efforts to solve the above-described problems, consequently, found that, when ultrasonic waves with a frequency of 0.1-5 MHz and an intensity of 200-800 mW/cm² which are controlled in an ultrasonic oscillation control unit are continuously irradiated into joint regions, the fundamental treatment of arthritis caused by damage to the joint cartilage becomes possible, thereby completing the present invention.

Accordingly, it is a main object of the present invention to provide an ultrasonic device for the treatment of arthritis, which can fundamentally treat arthritis using ultrasonic waves having a frequency of 0.1-5 MHz and an intensity of 200-800 mW/cm² and is easily operated.

To achieve the above object, the present invention provides an ultrasonic device for the treatment of arthritis, comprising: (a) ultrasonic oscillation control unit 130 for allowing the generation of ultrasonic waves with a frequency of 0.1-5 MHz and an intensity of 200-800 mW/cm²; (b) main control unit 300 for supplying voltage set by a user to a probe side for a predetermined time; (c) ultrasonic generator 330 for generating ultrasonic waves according to the control state of the main control unit; (d) ultrasonic vibrator 340 that vibrates according to ultrasonic waves inputted from the ultrasonic generator; and (e) probe 400 for directly transferring ultrasonic vibration generated from the ultrasonic vibrator to a joint to be treated, while being in contact with the ultrasonic vibrator.

The above and other objects, features and embodiments of the present invention will be more clearly understood from the following detailed description and the accompanying claims.

### BRIEF DESCRIPTION OF DRAWINGS

FIG. 1 shows a perspective view of an ultrasonic device for treating arthritis according to the prior art.

FIG. 2 shows an entire perspective view of an ultrasonic device for the treatment of arthritis according to one embodiment of the present invention.

FIG. 3 shows a block diagram for explaining the construction of ultrasonic device for the treatment of arthritis according to one embodiment of the present invention.

FIG. 4 shows proteoglycan concentration measured after arthritis-induced test animals were irradiated with ultrasonic waves using an ultrasonic device for the treatment of arthritis according to the present invention (C: a control group; HA: injected with hyaluronic acid; and US: ultrasonic irradiation).

FIG. 5 shows prostaglandin E2 (PGE2) concentration measured after arthritis-induced test animals were irradiated with ultrasonic waves using an ultrasonic device for the treatment of arthritis according to the present invention (C: a control group; HA: injected with hyaluronic acid; and US: ultrasonic irradiation).

FIG. 6 shows the expression levels of type 2 collagen (A) and MMP-3 (B) measured after arthritis-induced test animals were irradiated with ultrasonic waves using an ultrasonic device for the treatment of arthritis according to the present invention.

FIG. 7 is a graphic diagram showing observation results for the proliferation of cells according to the intensity of ultrasonic waves.

FIGS. 8A and 8B are graphic diagrams showing observation results for the proteoglycan synthesis of cells according to the intensity of ultrasonic waves.

FIG. 9 is a graphic diagram showing observation results for the synthesis and degradation of cellular collagen according to the intensity of ultrasonic waves.

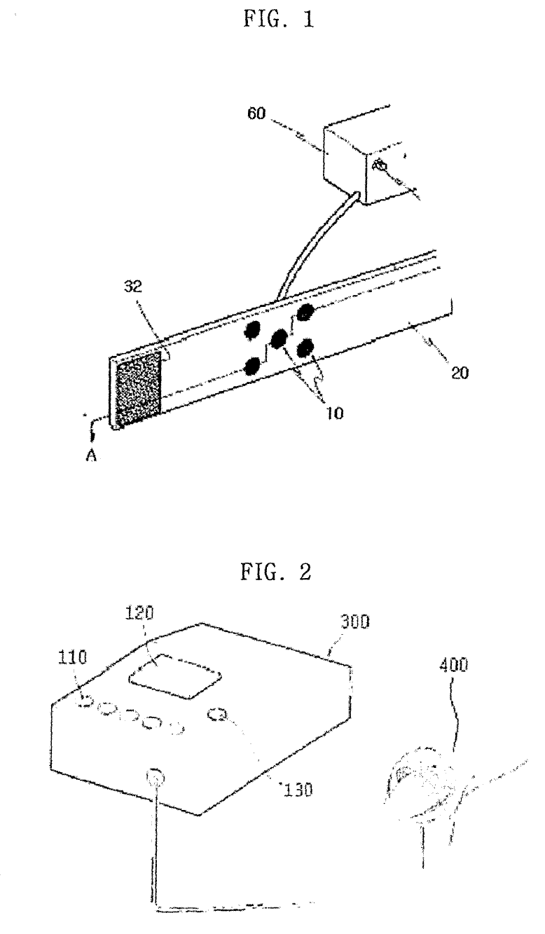

<Description of main reference numerals used in the drawing>

| | |
|---|---|
| 330: ultrasonic generator; | 300: main control unit; |
| 331: oscillator; | 332: dispenser; |
| 333: waveform determination unit; | 334: output amplification unit; |
| 335: output matching transformation unit; | |
| 340: ultrasonic vibrator; | 400: probe; |
| 110: power supply level controller; | 120: time setting unit; and |
| 140: ultrasonic oscillation control unit. | |

### DETAILED DESCRIPTION OF THE INVENTION

The present invention relates to an ultrasonic device for the treatment of arthritis, comprising: (a) ultrasonic oscillation control unit 130 for allowing the generation of ultrasonic waves with a frequency of 0.1-5 MHz and an intensity of 200-800 mW/cm²; (b) main control unit 300 for supplying voltage set by an user to a probe side for a predetermined time; (c) ultrasonic generator 330 for generating ultrasonic waves according to the control state of the main control unit; (d) ultrasonic vibrator 340 that vibrates according to ultrasonic waves inputted from the ultrasonic generator; and (e) probe 400 for directly transferring ultrasonic vibration generated from the ultrasonic vibrator to a joint to be treated, while being in contact with the ultrasonic vibrator.

In the present invention, the main control unit can be characterized by additionally comprising power supply level controller 110 or time setting unit 120, and the probe can be characterized by having an oval application end. Also, the oscillation control unit can be characterized in that it is so structured that it generates ultrasonic waves having a frequency of 0.5-2 MHz and an intensity of 300-600 mW/cm².

If ultrasonic waves which are irradiated for the treatment of arthritis have an intensity of less than 200 mW/cm², they fail to suitably stimulate cells and thus, have no effect of stimulating the proliferation of cells or increasing the synthesis of collagen and proteoglycan, which are the main components of an extracellular matrix. On the other hand, if ultrasonic waves have an intensity of more than 400 mW/cm², they have rather adverse effects on the formation of cartilage tissue, including an increase in cell death, reduction in the synthesis of proteoglycan and collagen, and an increase in the degradation of proteoglycan and collagen. Although the preferred intensity of ultrasonic waves was seen to be 100-400 mW/cm² in cell tests, preferred ultrasonic intensity for the treatment of arthritis is 200-800 mW/cm² because about 50% of ultrasonic waves are lost while being passed through the skin.

If ultrasonic waves have a frequency of less than 0.1 MHz, the vibrator increases in size to be in a bad contact with uneven joints, and if ultrasonic waves have a frequency of more than 5 MHz, their passage through articular skin and subcutaneous fat is limited.

Hereinafter, the present invention will be described in detail by the accompanying drawings.

FIG. 2 shows a perspective view of an ultrasonic device for arthritis treatment according to one embodiment of the present invention. As shown in FIG. 2, the inventive ultrasonic device has main control unit 300 at the outside and includes ultrasonic generator 330 and ultrasonic vibrator 340 at the inside. Since the main control unit includes power supply level controller 110, time setting unit 120 and an ultrasonic oscillation control unit 130, a user can optionally set the desired power supply level and operating time prior to the use of the arthritis treatment device. The ultrasonic oscillation control unit 130 is so set that it generates ultrasonic waves having a frequency of 0.1-5 MHz and an intensity of 200-800 mW/cm², which is the optimal conditions for arthritis treatment found by the present inventors, and more preferably ultrasonic waves having a frequency of 0.5-2 MHz and an intensity of 300-600 mW/cm².

The inventive ultrasonic device for arthritis treatment was so developed that it can be used by individuals at home. In view of safety, the inventive ultrasonic device is so set that its operation is automatically stopped by the time setting unit placed in the ultrasonic oscillation control unit as the treatment time lapses. It can be initially set to about 10 minutes and can be set up to 20 minutes.

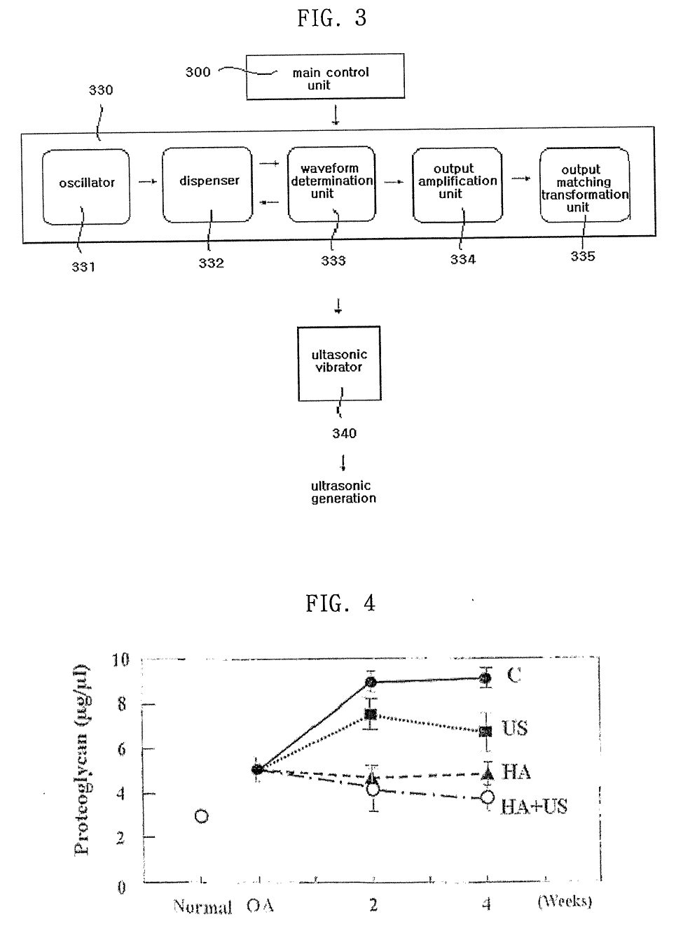

FIG. 3 is a block diagram for explaining the construction of the ultrasonic device for arthritis treatment according to one embodiment of the present invention. As shown in FIG. 3, the ultrasonic generator 330 includes oscillator 331, dispenser 332, waveform determination unit 333, output amplification unit 334, and output matching transformation unit 335. By this construction, ultrasonic waves inputted with data controlled in the main control unit to be generated from the ultrasonic generator 330 are applied to the ultrasonic vibrator 340 at the next position, and the ultrasonic vibrator 340 vibrates probe 400 which is in contact with it.

Specifically, the oscillator 331 is inputted with data controlled in the main control unit and generates fundamental ultrasonic waves which are used to generate ultrasonic waves to be finally determined, and the dispenser 332 is inputted with a waveform from the oscillator 331 and determines the state of dispense according to a state controlled in the main control unit of the ultrasonic device. The waveform determination unit 333 at the next position is inputted with ultrasonic waves which were dispensed and inter-controlled in the dispenser 332 and control state of main control unit 300, and it determines a final waveform to be used in the inventive ultrasonic device for arthritis treatment. This final waveform may be a continuous waveform or a pulse waveform.

The final ultrasonic waveform outputted from the waveform determination unit 333 is applied to the output amplification unit 334 at the next position, and the output amplification unit 334 amplifies the peak value of the determined ultrasonic waveform by the operation and power amplification of the ultrasonic waveform. Then, the amplified waveform outputted from the output amplification unit 334 is transformed by a matching and coupling transformer for final output in the output matching transformation unit 335 at the next position, and outputted in a state where it can be applied to the ultrasonic vibrator 340 at the next position.

The vibration of the ultrasonic vibrator 340 is transferred to the probe having an oval application end, and thus, the ultrasonic energy is transferred to cartilage cells deep in the skin's dermis which comes in contact with the probe. The transferred ultrasonic energy induces the regeneration of cartilage tissue by activating cartilage cells in the skin. Also, the continuous physical force of the ultrasonic waves having a low intensity of 100-400 mW/cm², and preferably, 150-250 mW/cm², activates cartilage tissue to induce the regeneration of the cartilage tissue, thus exhibiting the effect of fundamentally treating arthritis.

In use to obtain the preferred effect of the inventive ultrasonic device for arthritis treatment, a gel for ultrasonic treatment is sufficiently applied on a joint region to be treated, and then, the probe of the inventive device is lightly adhered to the side to which the gel has been applied. Once adhered, the probe is preferably not movable, and the inventive ultrasonic device is preferably used one or two times a day for less than 20 minutes each time, after the desired time is set using the time setting unit 120 of the main control unit.

The inventive ultrasonic device for the treatment of arthritis can be used at all regions with joints, including the knee (knee joint), the jaw, the shoulder, hips (hip joint), a finger, a toe, an elbow, a neck, and the vertebra.

As used herein, the term “ultrasonic waves” is a kind of sound and means ultrasonic waves of about 20,000 Hz or more, which are not heard by the ear of people.

### EXAMPLES

Hereinafter, the present invention will be described in more detail by examples. It is to be understood, however, that these examples are for illustrative purpose only and are not construed to limit the scope of the present invention.

### Example 1

To examine the effect of the ultrasonic device for arthritis treatment according to the present invention, the following test was performed using arthritis-induced animal models. In the test, in addition to carrying out ultrasonic irradiation, hyaluronic acid which is widely used as an osteoarthritis therapeutic agent was injected into the test animals. The hyaluronic acid is similar to the synovial fluid of joints, and thus, when it is injected into joints, it is effective in treating osteoarthritis because it allows joint cartilage to be protected by the recovery of lubricating action and is absorbed into the synovial fluid membrane or cartilage tissue to be able to stimulate the synthesis of hyaluronic acid by synovial fluid cells. When osteoarthritis develops, damage of joints becomes severe because the production of hyaluronic acid, which is a joint lubricating substance, decreases so that the joints fail to absorb or disperse an external impact. The hyaluronic acid used in the treatment of osteoarthritis is known to play an important role in that it allows the adhesion of proteoglycan known as the characteristic phenotype of cartilage cells (Swann, D. A., Biochem. Vitreou. Bull, Soc. Belg. Ophtalmol., 223:59, 1987).

### (1) Arthritis-Induced Animal Models

Sodium hyaluronate (LGCI, Taejeon, Korea) with an average molecular weight of 1,000 kDa was diluted to a concentration of 0.1%, and chemically fluorescence-labeled with fluroesceinamine (Sigma), and then, purified using 100% ethanol and saturated NaCl. As test animals, 18 New Zealand white female rabbits (Jung-Ang Experimental Animal Center, Korea) weighing 3.0 kg were used, and the irradiation of ultrasonic waves was performed by applying ultrasonic waves set to a frequency of 1 MHz and an intensity of 200 mW/cm² using the inventive ultrasonic device. Arthritis induction in the test animals was performed by inducing osteoarthritis in the knee joints of two hind legs of the test animals by ACLMT (anterior cruciate ligament and menisectomy transection) which is conventionally used (Jin, C. Z. et al., J. Korean Ortho. Res. Soc., 4:141, 2001).

### (2) Arthritis Therapeutic Effect by Ultrasonic Waves

0.5 ml of the fluorescence-labeled sodium hyaluronate was injected into the knee joints of hind legs of 18 New Zealand white female rabbits. Then, the right knees of all the test animals were irradiated with the above-described ultrasonic waves for 10 minutes every day, and in this ultrasonic treatment, exclusive gel for ultrasonic treatment was applied on the joint region such that the ultrasonic waves could be well transferred into the joint. The left knees of the test animals were not irradiated with ultrasonic waves and used as a control group.

To measure the concentrations of proteoglycan and prostaglandin E2 (PGE2) and the expression levels of type II collagen and MMP-3 (matrix metalloproteinase-3), the synovial fluid of the test animals was analyzed. The concentration of proteoglycan was measured by a 1,9-dimethylmethylene blue (DMB) dye-binding assay (Farndale, R. W. et al., Connect Tissue Res., 9:247, 1982), the concentration of PGE2 was measured using a PGE2 EIA (enzymeimmunoassy) system (Amersham Pharmacia Biotech, UK) after inducing osteoarthritis in the test animals, and the expression levels of type II collagen and MMP-3 were measured by Western blot.

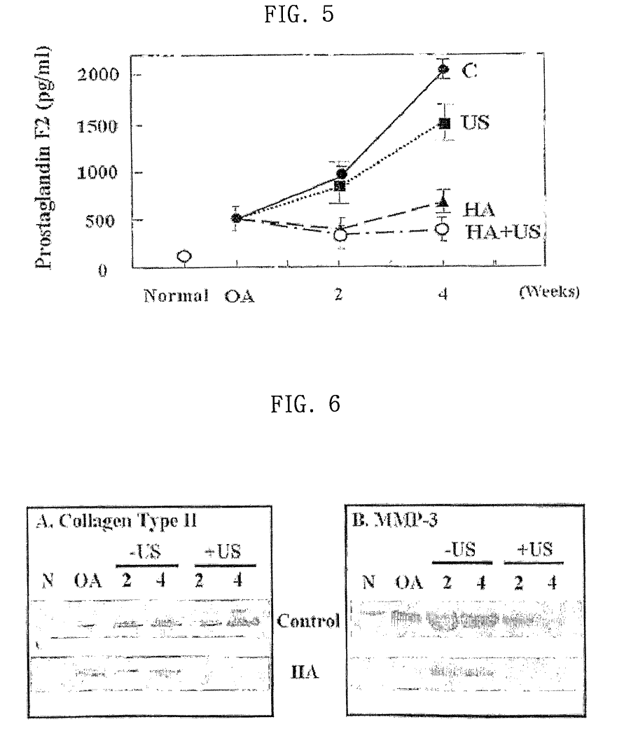

As a result, the test animals treated with both hyaluronic acid and ultrasonic waves showed the largest reduction in the secretion of synovial fluid in joints. Also, the test animals treated with both hyaluronic acid and ultrasonic waves showed not only a reduction in the amounts of proteoglycan and PGE2 in synovial fluid, which increase by the induction of osteoarthritis (FIG. 4 and FIG. 5), but also a reduction in the expression levels of type II collagen and MMP-3 (matrix metalloproteinase-3) caused by the induction of osteoarthritis (FIG. 6). These results suggest that the inventive ultrasonic device using low-intensity ultrasonic waves is effective in the treatment of arthritis.

### Example 2

To determine the optimal ultrasonic intensity, various tests were performed at a fixed frequency of 1 MHz while varying ultrasonic intensities. To examine the in vitro effect of ultrasonic waves on the proliferation and metabolism of cartilage cells, the following culture methods were used: an explant culture method of culturing cartilage tissue directly in human joints; a monolayer culture method of culturing cartilage cells isolated from cartilage tissue; and an alginate culture. The monolayer culture method is a culture method allowing the proliferation of cartilage cells to be effectively observed, the explant culture method is a culture method where it is easy to observe a cartilage matrix synthesized from cartilage cells, and the alginate culture method is a culture method where experiments on the matrix synthesis of cartilage cells and the maintenance of a cartilage cell phenotype are effective.

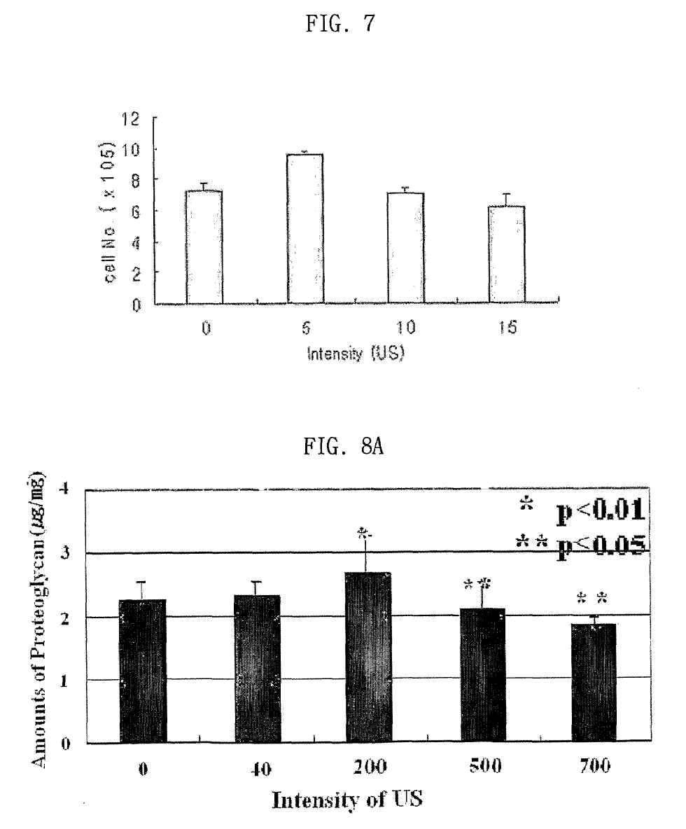

The effect of ultrasonic waves on the proliferation of cartilage cells was examined by the monolayer culture method, as a result, as shown in FIG. 7, the proliferation effect was shown to be the highest at irradiation intensity 5 (200-250 mW/cm²).

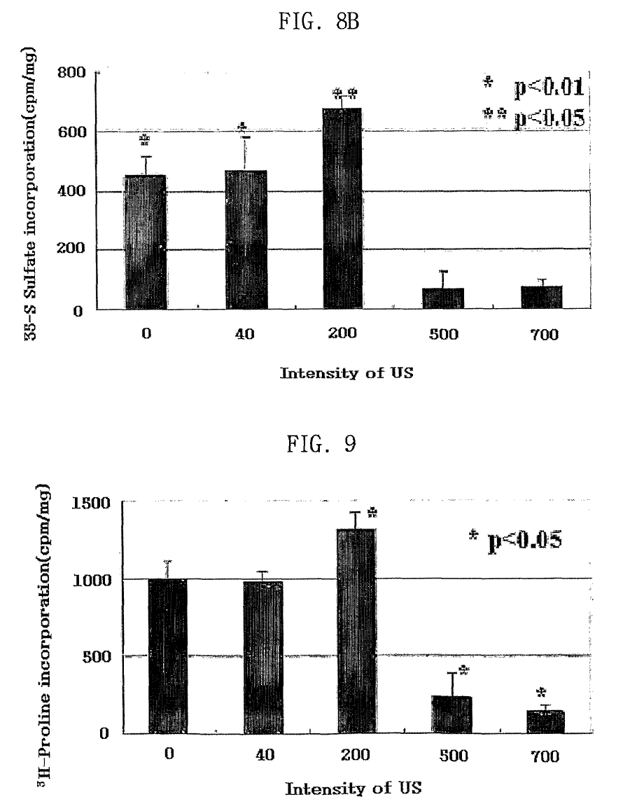

After ultrasonic irradiation into cartilage tissue, the amount of proteoglycan existing in the tissue was measured by the DMB assay, as a result, the amount of proteoglycan was the highest at an ultrasonic intensity of 200 mW/cm² and it was rather reduced at more than 700 mW/cm² (FIG. 8A). Also, because sulfate is needed for the synthesis of proteoglycan by cartilage cells, the proteoglycan synthesis rate of cartilage cells was analyzed by placing isotope-labeled sulfate into a culture broth and measuring the amount of sulfate absorbed into cartilage cells while irradiating the tissue with ultrasonic waves. As a result, the proteoglycan synthesis rate was statistically significantly increased at an ultrasonic intensity of 200 mW/cm², and it was rather reduced at 500 and 700 mW/cm² (FIG. 8B).

To examine the effect of ultrasonic waves on the synthesis of collagen among the matrices of cartilage cells, proline, which is a collagen synthesis material, was labeled with an isotope and placed in the culture broth, and the cartilage tissue was irradiated with ultrasonic waves. As a result, as shown in FIG. 9, the collagen synthesis rate was significantly increased at 200 mW/cm², and it was rather decreased at more than 500 mW/cm².

From the results of the cell tests, it could be seen that, although preferred ultrasonic intensity was 100-400 mW/cm², the preferred intensity of ultrasonic waves for use in the treatment of arthritis is 200-800 mW/cm² because about 50% of ultrasonic waves are generally lost while being passed through the skin.

### Example 3

### Clinical Test

A clinical test to evaluate the effectiveness of ultrasonic waves on the treatment of knee joint osteoarthritis was performed in 46 patients (40 females and 6 males). The selected test patients were patients having knee pain, who were diagnosed as knee joint osteoarthritis with a Kellgren-Lawrence radiographic grade of 1-3 as a result of radioactive testing, and patients with inflammatory arthritis, such as rheumatoid arthritis, were excluded from the selection.

In this clinical test, the frequency and ultrasonic intensity of the ultrasonic treatment device were set to about 1 MHz and about 400 mW/cm², respectively, and the treatment time was set to 20 minutes. Two sites (left and right sides) on one knee were selected and applied sufficiently with gel for ultrasonic treatment, and the probe was lightly adhered to the affected parts, and an operation switch was pressed to initiate treatment. For the evaluation of effectiveness, WOMAC survey (evaluations for pains, stiffness and function) was performed, and the treatment satisfaction in patients, and the treatment effect by test doctors, were evaluated. For clinical test, patient's joint synovial fluid was extracted before and after treatment, and measured for synovial fluid volume, the concentration of PGE2 and the amount of total protein in the synovial fluid.

### (1) Observation Testing Method

Joint function test: before treatment and at 3, 5 and 8 weeks of treatment, scores in each of test items (pain, stiffness and function) were recorded in total using WOMAC survey (survey questionnaire).

Treatment satisfaction: at 3, 5 and 8 weeks, the treatment effect in the subject patients, was recorded by a questionnaire.

At 8 weeks, the treatment effect was evaluated and recorded by a person in charge of clinical test by collectively examining the results of joint function test, treatment satisfaction and synovial fluid examination before and after treatment.

### (2) Evaluation Criterion, Evaluation Method and Analytical Method

Evaluation criterion: the case where the evaluation score for treatment effect in the subject patients after completion of the treatment has been rated as 1 or more based on a case report form prepared for each of the patients, by the person in charge of clinical test, was determined to have effectiveness.

Evaluation method and analytical method: the results of changes in joint function test and synovial fluid examination before and after treatment were statistically analyzed by paired t-test, and the case of p<0.0⁵ was determined to be statistically significant. The treatment rate (%) was calculated by the following equation:

Treatment rate(%)(the number of subject patients having a treatment effect evaluation score of 1 or more/the number of total subject patients)×100

### (3) Results of Clinical Test Scores

After completion of 8 weeks of clinical test, WOMAC survey was carried out, as a result, all test items, including pain, stiffness and function, were significantly reduced, and this reduction showed a significant difference according to first, second and third interviews. Also, during a clinical test period of 8 weeks, there was no case of a side effect occurred in the subject patients.

The volume of synovial fluid and the amount of total protein showed a positive correlation with an increase and decrease in WOMAC score, and the concentration of prostaglandin showed a negative correlation but had no statistically significant difference. An increase and decrease in the detailed WOMAC items (pain, stiffness and function) and an increase and decrease in most significant finding parameters for synovial fluid were analyzed, as a result, the amount of total protein showed a positive correlation with the amount of pains, and the others had no significant difference.

After completion of the clinical test, the treatment effect was evaluated by a person in charge of the clinical test by collectively examining the results of joint function test, treatment satisfaction and synovial fluid examination before and after treatment. The results were as follows: 4 persons (8.7%) for a treatment effect score of −1 at 8 weeks; none for a score of zero; 16 persons (34.8%) for a score of 1; 26 persons (56.5%) for a score of 2; and none for a score of 3.

In conclusion, the results of joint function test by WOMAC survey showed that 42 persons among a total of 46 persons were improved, indicating a treatment rate of 91.3%, and the treatment satisfaction index was 89.2%.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

As described in detail above, the ultrasonic device according to the present invention is so constructed that it generates ultrasonic waves with an intensity of 200-800 mW/cm² and applies low-intensity ultrasonic energy of 100-400 mW/cm² to cartilage cells deep in the skin's dermis. Thus, the inventive ultrasonic device can fundamentally treat degenerative arthritis, since it induces the regeneration of cartilage tissue by the activation of cartilage tissue. Also, the inventive ultrasonic device can be conveniently used by simple operation at home and in the offices.

1. An ultrasonic device for the treatment of arthritis, comprising: (a) an ultrasonic oscillation control unit for allowing the generation of ultrasonic waves with a frequency of 0.1-5 MHz and an intensity of 200-800 mW/cm²; (b) a main control unit for supplying voltage set by a user to a probe side for a predetermined time; (c) an ultrasonic generator for generating ultrasonic waves according to the control state of the main control unit; (d) an ultrasonic vibrator that vibrates according to ultrasonic waves inputted from the ultrasonic generator; and (e) a probe for directly transferring ultrasonic vibration generated from the ultrasonic vibrator to a joint to be treated, while being in contact with the ultrasonic vibrator.

2. The ultrasonic device for the treatment of arthritis according to claim 1, wherein the oscillation control unit is adapted to generate ultrasonic waves having a frequency of 0.5-2 MHz and an intensity of 300-600 mW/cm².

3. The ultrasonic device for the treatment of arthritis according to claim 1, wherein the main control unit further comprises a power supply level controller or a time setting unit.

4. The ultrasonic device for the treatment of arthritis according to claim 1, wherein the probe has an oval application end.
